(19) United States
(12) Patent Application Publication
(10) Pub. No.: US 2016/0135752 A1
(43) Pub. Date: May 19, 2016

(54) SMALL DATA AGGREGATOR FOR PERSONAL HEALTH MANAGEMENT

(71) Applicant: Lenovo (Singapore) Pte. Ltd., Singapore (SG)

(72) Inventor: Suzanne Marion Beaumont, Wake Forest, NC (US)

(21) Appl. No.: 14/547,856

(22) Filed: Nov. 19, 2014

(51) Int. Cl. A61B 5/00; A61B 5/117; A61B 5/11

(52) U.S. Cl. CPC A61B 5/6898; A61B 5/1118; A61B 5/4812; A61B 5/4866; A61B 5/0022; A61B 5/117; A61B 5/7282; A61B 5/1112

(57) ABSTRACT

One embodiment provides a method, including: identifying, using a processor of an electronic device, at least one user associated with a user of the electronic device; receiving, using a network component of the electronic device, health-related information associated with the at least one user; generating, using a processor of the electronic device, a notification based on the health-related information; and providing, using an output device of the electronic device, the notification to the user of the electronic device. Other aspects are described and claimed.

### BACKGROUND

When a person gets sick (and/or others associated with that person, e.g., family, friends, co-workers, etc.), questions often arise regarding what is going on generally (e.g., is something “going around”), how was the illness contracted, who was the illness caught from, how long will the illness last, can the illness be spread to others, how serious is the illness, etc. Such information might be helpful in avoiding illnesses and/or estimating care needs once an illness has been contracted.

While some public health information is available (e.g., from local and national health care associations or government agencies), such information tends to be impersonal, i.e., dealing with serious public health issues such as epidemics of massive proportions, not smaller scale illness outbreaks among those closest to a person (e.g., friends, family, co-workers, neighbors, members of a sports team, etc.). Similarly, a person that becomes sick can contact his or her doctor, but this often occurs after the fact (the illness has already been contracted), often involves incomplete information (e.g., who else has the illness), and often involves a series of phone calls back and forth before an actual conversation takes place or meaningful information is exchanged.

### BRIEF SUMMARY

In summary, one embodiment provides a method, comprising: identifying, using a processor of an electronic device, at least one user associated with a user of the electronic device; receiving, using a network component of the electronic device, health-related information associated with the at least one user; generating, using a processor of the electronic device, a notification based on the health-related information; and providing, using an output device of the electronic device, the notification to the user of the electronic device.

Another aspect provides an electronic device, comprising: an output device; a network component; a processor; and a memory storing instructions executable by the processor to: identify at least one user associated with a user of the electronic device; receive, using the network component of the electronic device, health-related information associated with the at least one user; generate a notification based on the health-related information; and provide, using the output device, the notification to the user of the electronic device.

A further aspect provides a product, comprising: a storage device having code stored therewith, the code being executable by a processor and comprising: code that identifies at least one user associated with a user of an electronic device; code that receives health-related information associated with the at least one user; code that generates a notification based on the health-related information; and code that provides the notification to the user of the electronic device.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates an example of information handling device circuitry.

FIG. 2 illustrates another example of information handling device circuitry.

FIG. 3 illustrates an example of small data aggregation for personal health management.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

Because public health information is often lacking in detail, an embodiment aggregates data from a smaller group that is contextually relevant and provides health-related data regarding the same to group members. In essence, an embodiment provides data similar to a public health agency, e.g., the Center for Disease Control (CDC), but on a personalized, smaller scale. An embodiment provides tracking of health-related information, e.g., directly from enrolled sensors/Internet of Things (IoT) devices and/or from self-reporting by group members, and associates this health-related information with relevant groups whose context makes the information more useable and understandable.

The groupings may include groups generated or derived from a variety of sources, e.g., a device contact list, a calendar application, actual locations visited, etc. Groupings may also include larger groups to which a user has a weaker association, such as a doctor's or a practice's patient list, a physical neighborhood, a physical town or the like. Groupings may also include smaller groups with stronger associations, such as a user's classmates, those on a particular sports team, a volunteer group to which the user belongs, a work group, etc.

Health-related data may be input by the users of the group directly or sensed data may be processed into health-related information. Sensor readings may include body temperature, activity level, sleep cycle, a chemical reaction (such as a material that changes color based on body temperature, respiration, perspiration, etc.) or any other sensor data that can paint a picture of a person's normal self versus a self that is out of the ordinary, such as when a person is sick. Other personal heath measures may include heart rate, blood pressure, blood sugar level or the like. Depending on the devices and sensors available, e.g., via wearable devices and/or home-based health monitoring devices, the quality and nature of health related data available may differ. In this regard, an embodiment may employ pattern analysis and machine learning to estimate a user's condition.

Sensors may include smart home sensors that indicate when a person is not well. For example, a smart thermostat may indicate when a user raises the temperature at a non-traditional time—perhaps indicating that person has the chills. Other smart devices may indicate when a person is not doing activities that he or she normally would and/or is using resources that are out of that person's normal habit. Such data may indicate that perhaps the person is feeling under the weather by missing meetings, visiting the doctor or pharmacy, etc.

Other data may be collected and used to generate health-related information, including refining, sorting or distilling other collected data. For example, location information could be used to alert a user or others to the risk of exposure to reported symptoms, e.g., when traversing certain routes or visiting certain venues.

Given the availability of inputs, an embodiment may generate and provide a rich source of health-related information regarding a personalized group. Thus, it may be possible to pin point who represents a risk and/or where an illness might be occurring, or may provide a peer group for a set of symptoms from which a user may learn the illness duration and severity, care and treatment, etc.

For some groups or types of health-related information, individuals may want to preserve anonymity, for example within a larger community. For some groups, users may have no choice but to remain anonymous, for example if the information is derived from an anonymous source such as statistical information derived from a doctor's patient list, a public health database reporting illness occurrences, etc. On the other hand, e.g., for smaller groups, users may want to publicize their identity and offer detailed health-related information, for example participants in a youth soccer team may opt into sharing their health related information such that team members remain apprised of each other's conditions, duration of illnesses, expected clearance times of an illness, etc. Policies thus may be crafted to include or exclude identifying or other information, as well as allowing users to opt into and out of reporting.

An embodiment thus provides access to information organized in a manner that is relevant to particular users such that these users can manage their health in the smartest way possible.

The illustrated example embodiments will be best understood by reference to the figures. The following description is intended only by way of example, and simply illustrates certain example embodiments.

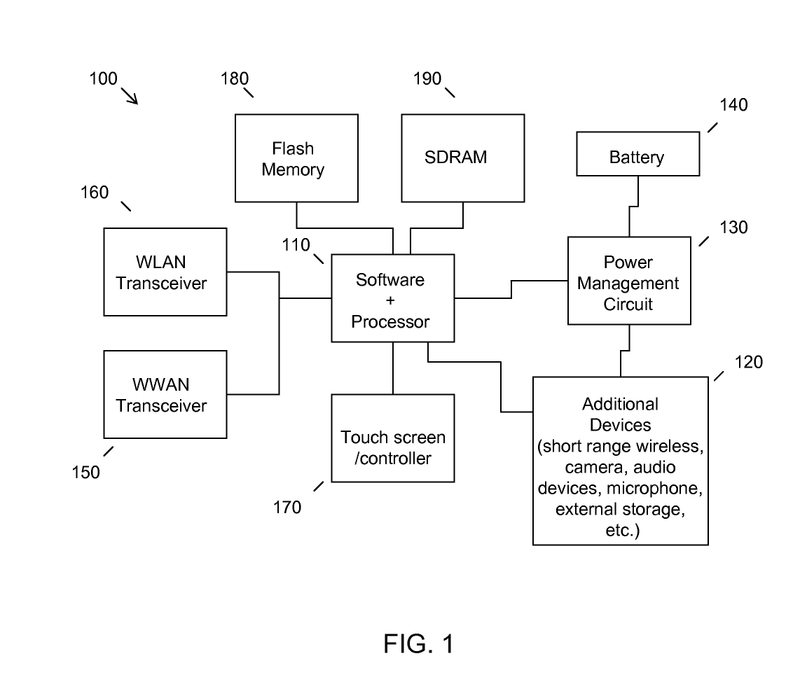

While various other circuits, circuitry or components may be utilized in information handling devices, with regard to smart phone and/or tablet circuitry 100, an example illustrated in FIG. 1 includes a system on a chip design found for example in tablet or other mobile computing platforms.

Software and processor(s) are combined in a single chip 110. Processors comprise internal arithmetic units, registers, cache memory, busses, I/O ports, etc., as is well known in the art. Internal busses and the like depend on different vendors, but essentially all the peripheral devices (120), for example a keyboard, camera, microphone, and the like, may attach to a single chip 110. The circuitry 100 combines the processor, memory control, and I/O controller hub all into a single chip 110. Also, systems 100 of this type do not typically use SATA or PCI or LPC. Common interfaces, for example, include SDIO and I2C.

There are power management chip(s) 130, e.g., a battery management unit, BMU, which manage power as supplied, for example, via a rechargeable battery 140, which may be recharged by a connection to a power source (not shown). In at least one design, a single chip, such as 110, is used to supply BIOS like functionality and DRAM memory.

System 100 typically includes one or more of a WWAN transceiver 150 and a WLAN transceiver 160 for connecting to various networks, such as telecommunications networks and wireless Internet devices, e.g., access points. Additional devices 120 are commonly included, e.g., such as a camera, microphone, audio output device, or other input/output devices. System 100 often includes a touch screen 170 for data input and display/rendering. System 100 also typically includes various memory devices, for example flash memory 180 and SDRAM 190.

In some examples, circuitry such as that outlined in FIG. 1 may include additional devices 120 such as radios or other communications devices that connect to a variety of networks, including ad-hoc personal area networks (PAN's), for participation in an ad-hoc network of devices communicating over short range, using radios such as BLUETOOTH radios, BLUETOOH LOW ENERGY radios, etc.) or even body area networks (BAN's), which are comprised of an ad-hoc network of wearable devices.

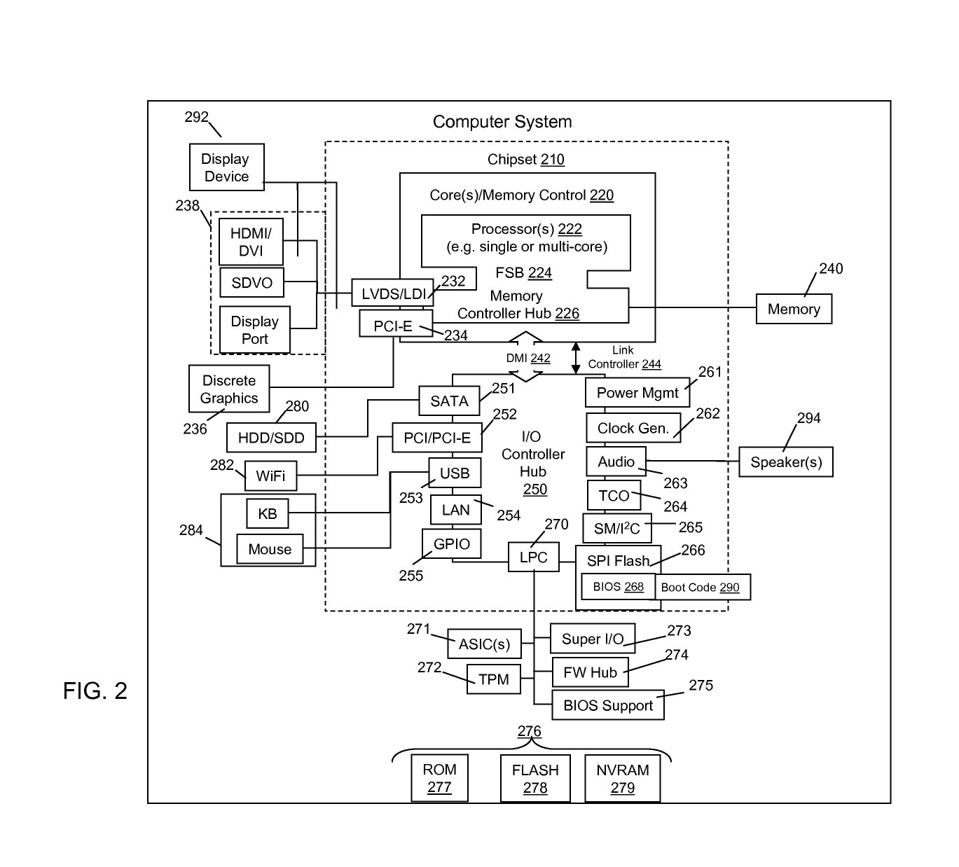

FIG. 2 depicts a block diagram of another example of information handling device circuits, circuitry or components. The example depicted in FIG. 2 may correspond to computing systems such as the THINKPAD series of personal computers sold by Lenovo (US) Inc. of Morrisville, N.C., or other devices. As is apparent from the description herein, embodiments may include other features or only some of the features of the example illustrated in FIG. 2.

The example of FIG. 2 includes a so-called chipset 210 (a group of integrated circuits, or chips, that work together, chipsets) with an architecture that may vary depending on manufacturer (for example, INTEL, AMD, ARM, etc.). INTEL is a registered trademark of Intel Corporation in the United States and other countries. AMD is a registered trademark of Advanced Micro Devices, Inc. in the United States and other countries. ARM is an unregistered trademark of ARM Holdings plc in the United States and other countries. The architecture of the chipset 210 includes a core and memory control group 220 and an I/O controller hub 250 that exchanges information (for example, data, signals, commands, etc.) via a direct management interface (DMI) 242 or a link controller 244. In FIG. 2, the DMI 242 is a chip-to-chip interface (sometimes referred to as being a link between a “northbridge” and a “southbridge”). The core and memory control group 220 include one or more processors 222 (for example, single or multi-core) and a memory controller hub 226 that exchange information via a front side bus (FSB) 224; noting that components of the group 220 may be integrated in a chip that supplants the conventional “northbridge” style architecture. One or more processors 222 comprise internal arithmetic units, registers, cache memory, busses, I/O ports, etc., as is well known in the art.

In FIG. 2, the memory controller hub 226 interfaces with memory 240 (for example, to provide support for a type of RAM that may be referred to as “system memory” or “memory”). The memory controller hub 226 further includes a LVDS interface 232 for a display device 292 (for example, a flat panel display, touch screen, etc.). A block 238 includes some technologies that may be supported via the LVDS interface 232 (for example, serial digital video, HDMI/DVI, display port). The memory controller hub 226 also includes a PCI-express interface (PCI-E) 234 that may support discrete graphics 236.

In FIG. 2, the I/O hub controller 250 includes a SATA interface 251 (for example, for HDDs, SDDs, etc., 280), a PCI-E interface 252 (for example, for wireless connections 282), a USB interface 253 (for example, for devices 284 such as a digitizer, keyboard, mouse, camera, phone, microphone, storage, other connected devices, etc.), a network interface 254 (for example, LAN), a GPIO interface 255, a LPC interface 270 (for ASICs 271, a TPM 272, a super I/O 273, a firmware hub 274, BIOS support 275 as well as various types of memory 276 such as ROM 277, Flash 278, and NVRAM 279), a power management interface 261, a clock generator interface 262, an audio interface 263 (for example, for speakers 294), a TCO interface 264, a system management bus interface 265, and SPI Flash 266, which can include BIOS 268 and boot code 290. The I/O hub controller 250 may include gigabit Ethernet support.

The system, upon power on, may be configured to execute boot code 290 for the BIOS 268, as stored within the SPI Flash 266, and thereafter processes data under the control of one or more operating systems and application software (for example, stored in system memory 240). An operating system may be stored in any of a variety of locations and accessed, for example, according to instructions of the BIOS 268. As described herein, a device may include fewer or more features than shown in the system of FIG. 2.

Device circuitry, as for example outlined in FIG. 1 and/or FIG. 2, may be used in user electronic devices such as laptops, cellular phones/smart phones, tablet computing devices or other user electronic devices. Additionally, as described herein, various smart devices (e.g., in-home smart devices, devices provide in cars, etc.) may include some or all of the components outlined in FIG. 1 and/or FIG. 2. Such devices may include environmental sensors that report sensed data that may be used, as further elaborated on herein, to generate health-related data regarding users in a particular group. Such devices may include wearable devices, which, at this point in time, may communicate with other devices (e.g., phones, tablets, etc.) to upload, or share, or otherwise communicate their data to other devices, including an aggregator such as implemented in a cloud device or coordinating device. For example, such devices may participate and to communicate through ad-hoc networks such as PAN's and BAN's.

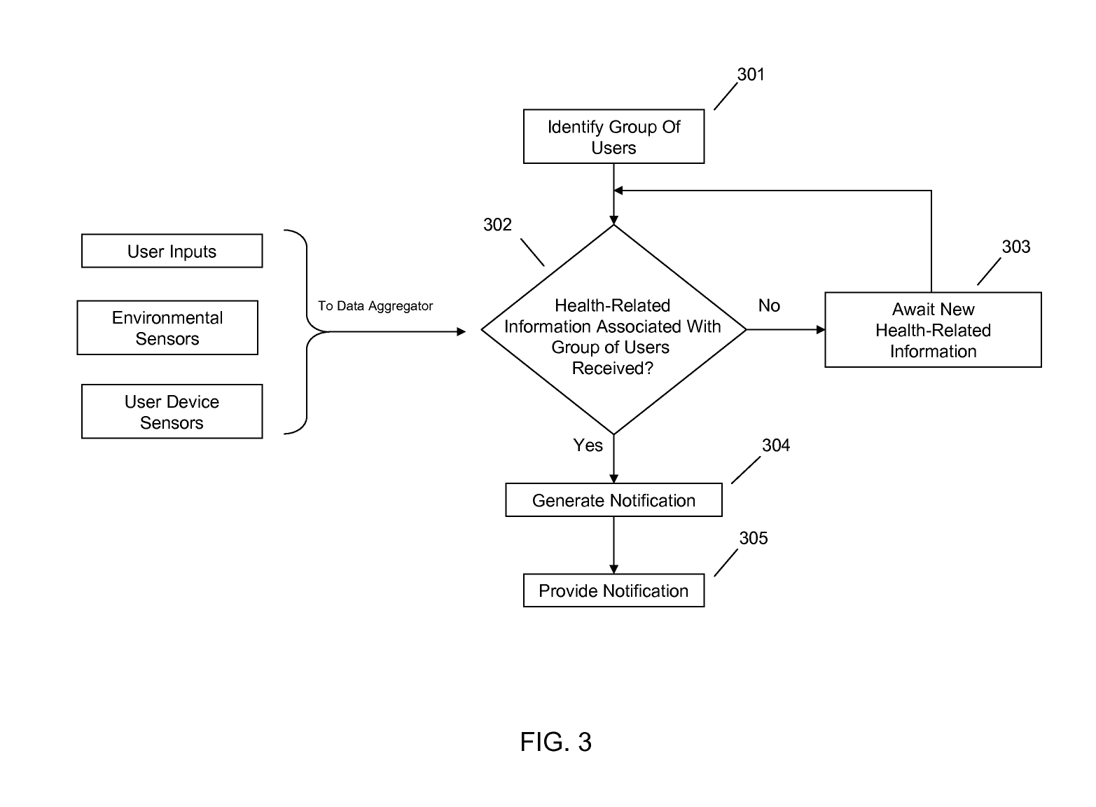

As illustrated in the non-limiting example of FIG. 3, an embodiment provides a method of aggregating data from a small group for personal health management. In the illustrated example, an embodiment identifies a group of users associated with a user of an electronic device at 301. For example, if downloaded as a smart phone application, the data aggregator permits a user of the smart phone to identify a group for which health-related information should be aggregated and reported on. As further explained, this group may include the user of the smart phone and his or her health related data may be reported to others within the group.

A grouping of users may be identified at 301 using automatic discovery techniques and/or manual input by the user. For example, a small, personalized group may be selected using a contact list of the smart phone (i.e., users listed in a contacts list). The contact list includes at minimum users with which the user of the smart phone or device has chosen for inclusion in a short list of contacts, making these users candidates for inclusion in data aggregation for health care monitoring.

Similarly, other on-device information may prove useful in identifying a group of users. For example, a calendar appointment stored on the smart phone may include user identifying information, e.g., co-workers scheduled to attend a work meeting. These users may be included in a work group that is associated with the user of the device. Likewise, other data may act as a source of data for indentifying users at 301. For example, a group of users may be associated with a geographic location, such as classmates at a school where the user device has been according to GPS location data, etc.

After a group of users has been identified at 301, an embodiment may receive health-related information associated with at least one of the group of users at 302. For example, if a device contacts list has been used to create a group at 301, e.g., a family group, these users may opt-in to sharing health-related information with others in the group. This opt-in process may take a variety of forms, for example users may opt in to sharing health related information by manually agreeing to provide such data to particular users or groups of users, as further described herein.

If it is determined that health-related data has been received at 302, an embodiment may generate a notification at 304 regarding the health-related information. By way of example, if a user in the family group contracts a cold, this may be reported by that user, e.g., by manually entering this information into an application running on one of that user's personal electronic devices. Similarly, that a user in the family group has contracted a cold might be inferred, e.g., from a that user visiting a particular location (pharmacy, doctor's office, etc.) along with other information, e.g., social media posting, body temperature data, or other data associated with that user and available to the application, either directly or indirectly.

The notification generated at 304 may take a variety of forms. For example, the notification may include a visual graphic or other data that may be presented or provided to the user at 305. By way of example, the notification may be provided at 305 automatically, e.g., substantially immediately after a new notification is generated at 304. Alternatively, the notification may be provided at 305 in response to a user action, e.g., a user opening his or her application, requesting an update.

In terms of the content of the notification, in an embodiment the notification comprises an indication that at least one of the users in the group is ill. This may include specific details regarding the illness, e.g., date and time of the illness beginning, the actual identification of the illness, symptoms the user is experiencing (e.g., increased temperature, reduced or increased sleep times, missed work or school day, etc.), some of which may depend on the nature of data available and/or the ill user's willingness to share.

For example, an ill user may have data related to his or her illness sensed, e.g., using a sensor of his or her electronic device or other smart device (e.g., smart thermostat or environmental sensor sensing ambient temperature and making inferences about the user, a smart thermometer that reports actual measured temperature, etc.). This data may be stored by the electronic device that senses it and reported to other devices of users in the small group. Thus for example the health-related data associated with a user may include body temperature data, activity level data, meeting attendance data, sleep cycle data, dietary data, etc. This data may be communicated to a remote device, e.g., another user device within the group.

As may be appreciated, users should retain control over which groups they belong to and which data is shared. Thus, an embodiment provides an interface with which a user may input reporting configuration data. This reporting configuration data may then be translated, e.g., according to a reporting policy, into a control for what, if any, health related data is communicated to other users and/or to which group(s).

There may be many scenarios where a user wishes to receive health-related data proactively. For example, a user might wish to receive a notification that is proactively communicated based on particular parameters. By way of non-limiting example, a user might wish to be apprised that another group member has become ill prior to encountering that group member. By way of specific example, if a family member falls ill, another family member may wish to be apprised of this information proactively, e.g., prior to a visit, such that he or she may take appropriate precautions. Likewise, working groups, sports teams, etc., may use such health-related information to some advantage, e.g., reducing the spread of illness, receiving symptom information, and/or receiving other information (incubation time, time to recovery, etc.).

The various embodiments thus permit a user to share health-related data with a small group in a personalized way. The nature and type of health-related information shared between group members, and in fact even the size or membership of the group, is configurable such that users remain in control of their health information. This also permits increased sharing, e.g., via making anonymous certain identifying information. By way of example, a neighborhood group may report illnesses experienced generally by members but not identify which specific members have fallen ill. This allows the others in the neighborhood to be apprised that certain neighbors are ill, but not necessarily which ones.

Additionally, other sources of information may be used by the various embodiments. For example, if a group member identifies that he or she has influenza, this information may be communicated to the group members, along with a link to additional information, e.g., general information regarding influenza, its nature and duration, best practices for avoiding its contraction, locations offering vaccination against the same, etc.

An embodiment thus facilitates a user's ability to remain apprised of health-related information in a way that promotes health. The nature of this information sharing is, in one example, personalized to a small group such that the users of the group only receive information that is of use to them. That is, rather than accessing community wide data alone, which may not be particularly helpful, an embodiment facilitates sharing of particularized health-related information that is of personal use to the group members, permitting them to be aware of the other member's health status, location, and make choices on the basis thereof.

As will be appreciated by one skilled in the art, various aspects may be embodied as a system, method or device program product. Accordingly, aspects may take the form of an entirely hardware embodiment or an embodiment including software that may all generally be referred to herein as a “circuit,” “module” or “system.” Furthermore, aspects may take the form of a device program product embodied in one or more device readable medium(s) having device readable program code embodied therewith.

It should be noted that the various functions described herein may be implemented using instructions stored on a device readable storage medium such as a non-signal storage device that are executed by a processor. A storage device may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random access memory (RAM), a read-only memory (ROM), an erasable programmable read-only memory (EPROM or Flash memory), a portable compact disc read-only memory (CD-ROM), or any suitable combination of the foregoing. In the context of this document, a storage device is not a signal and “non-transitory” includes all media except signal media.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the figures, which illustrate example methods, devices and program products according to various example embodiments. It will be understood that the actions and functionality may be implemented at least in part by program instructions. These program instructions may be provided to a processor of a general purpose device, a special purpose device, or other programmable data processing device to produce a machine, such that the instructions, which execute via a processor of the device implement the functions/acts specified.

It is worth noting that while specific blocks are used in the figures, and a particular ordering of blocks has been illustrated, these are non-limiting examples. In certain contexts, two or more blocks may be combined, a block may be split into two or more blocks, or certain blocks may be re-ordered or re-organized as appropriate, as the explicit illustrated examples are used only for descriptive purposes and are not to be construed as limiting.

As used herein, the singular “a” and “an” may be construed as including the plural “one or more” unless clearly indicated otherwise.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A method, comprising:
identifying, using a processor of an electronic device, at least one user associated with a user of the electronic device;
receiving, using a network component of the electronic device, health-related information associated with the at least one user;
generating, using a processor of the electronic device, a notification based on the health-related information; and
providing, using an output device of the electronic device, the notification to the user of the electronic device.

2. The method of claim 1, wherein the at least one user is identified using data selected from the group consisting of a contact list of the electronic device, a calendar appointment of the electronic device, and geographic location data.

3. The method of claim 1, wherein the notification comprises an indication that the at least one user is ill.

4. The method of claim 1, further comprising:
sensing, using a sensor, health-related data associated with the user of the electronic device; and
communicating, using a network component of the electronic device, the health-related data associated with the user of the electronic device to a remote device.

5. The method of claim 4, wherein the health-related data associated with the user is selected from the group consisting of activity level data, meeting attendance data, sleep cycle data, data derived from a wearable device, and dietary data.

6. The method of claim 4, further comprising storing, using a memory of the electronic device, the health-related data associated with the user of the electronic device.

7. The method of claim 4, further comprising:
receiving, using an input device of the electronic device, reporting configuration data; and
configuring, using a processor, the communicating based on the reporting configuration data.

8. The method of claim 1, further comprising:
receiving, from another electronic device, health-related data associated with the user of the electronic device.

9. The method of claim 8, wherein the receiving health-related data associated with the user of the electronic device comprises receiving data derived from an environmental sensor.

10. The method of claim 1, wherein the notification is proactively communicated to the user of the electronic device.

11. An electronic device, comprising:
an output device;
a network component;
a processor; and
a memory storing instructions executable by the processor to:
identify at least one user associated with a user of the electronic device;
receive, using the network component of the electronic device, health-related information associated with the at least one user;
generate a notification based on the health-related information; and
provide, using the output device, the notification to the user of the electronic device.

12. The electronic device of claim 11, wherein the at least one user is identified using data selected from the group consisting of a contact list of the electronic device, a calendar appointment of the electronic device, and geographic location data.

13. The electronic device of claim 11, wherein the notification comprises an indication that the at least one user is ill.

14. The electronic device of claim 11, wherein the instructions are further executable by the processor to:
sense, using a sensor, health-related data associated with the user of the electronic device; and
communicate, using the network component, the health-related data associated with the user of the electronic device to a remote device.

15. The electronic device of claim 14, wherein the health-related data associated with the user is selected from the group consisting of activity level data, meeting attendance data, sleep cycle data, data derived from a wearable device, and dietary data.

16. The electronic device of claim 14, wherein the instructions are further executable by the processor to store, using a memory of the electronic device, the health-related data associated with the user of the electronic device.

17. The electronic device of claim 14, wherein the instructions are further executable by the processor to:
receive, using an input device of the electronic device, reporting configuration data; and
configure the communicating based on the reporting configuration data.

18. The electronic device of claim 11, wherein the instructions are further executable by the processor to:
receive, from another electronic device, health-related data associated with the user of the electronic device.

19. The electronic device of claim 18, wherein to receive health-related data associated with the user of the electronic device comprises receiving data derived from an environmental sensor.

20. A product, comprising:
a storage device having code stored therewith, the code being executable by a processor and comprising:
code that identifies at least one user associated with a user of an electronic device;
code that receives health-related information associated with the at least one user;
code that generates a notification based on the health-related information; and
code that provides the notification to the user of the electronic device.
